(19) United States
(12) Patent Application Publication
(10) Pub. No.: US 2022/0183695 A1
(43) Pub. Date: Jun. 16, 2022

(54) OCCLUDING MEDICAL DEVICES AND METHODS OF USE

(71) Applicant: Richard D. Julason, JR., New York, NY (US)

(72) Inventor: Richard D. Julason, JR., New York, NY (US)

(21) Appl. No.: 17/550,623

(22) Filed: Dec. 14, 2021

Related U.S. Application Data
(60) Provisional application No. 63/125,325, filed on Dec. 14, 2020.

(51) Int. Cl. A61B 17/12

(52) U.S. Cl. CPC A61B 17/12036; A61B 17/12109; A61B 17/12131

(57) ABSTRACT

Medical devices and methods for occluding fluid flow in a blood vessel, and specifically for embolizing vessels concentrically, are provided. In an embodiment, the medical devices comprise a proximal end, a distal end, an elongated member positioned between the proximal end and the distal end, wherein the elongated member has a lumen passing through, and a flow-limiting member coupled to the distal end.

### FIELD OF THE DISCLOSURE

The present disclosure relates to implantable and expandable medical devices suitable for occluding fluid flow in a blood vessel. In particular, the present disclosure relates to occlusion devices having an interwoven support structure and a fluid flow-occluding membrane.

### BACKGROUND

Generally, embolization or occlusion of a vascular vessel, an artery or vein, is done intentionally for a variety of reasons, including treatment of extravascular perfusion (hemorrhage), de-vascularize a targeted area such as a tumor or arterio-venous malformation (AVM) prior to surgical resection in order to limit blood loss during surgery, or to alter blood flow or drainage patterns to treat a chronic condition. There are two types of embolization procedures: terminal vessel (e.g., particle embolization) and targeted embolization (coil embolization).

Terminal vessels are often treated with tiny particles (20-1000 microns) which are delivered in a flow directed manner via a microcatheter. It is as simple as navigating the tip of the microcatheter to the desired location, injecting the particles, and wherever the blood flow takes them is wherever they end up. A shotgun approach if you will. Targeted vessel sacrifices or embolization are more precise due to potential complications associated with being “non-targeted.” For instance, if the embolic device migrates further out (distal) or too far towards the delivery catheter (proximal), the operator may inadvertently embolize a vessel that needed to remain open (patent). Since the inception of embolization, targeted embolization has been conducted in a longitudinal manner working from distal to proximal.

Some of the earliest targeted embolization devices were stainless steel coils that resemble a corkscrew. Over time, “coil” technology has improved with the advent of platinum coils that are MRI compatible and finally detachable coils that provided increased safety. However, the technique has largely remained the same: start as distal as possible and coil backwards until blood flow no longer continues past the coils. An alternative device to the coil is a vascular plug, which consists of a metal scaffold designed to halt blood flow or “plug” the vessel. Conceptually, vascular plugs work in the same technical manner of longitudinal embolization by working in a distal to proximal plane.

However, these devices experience some shortcomings. Despite being the only endovascular technique for as long these procedures have been performed, there are significant limitations to this approach with these devices. First, if the coils (or plugs) migrate further out (distally), the vessel will be occluded at a level that was not desired and can cause patient complications. Second, the coils can migrate proximally and occlude larger, non-targeted vessels that can also lead to patient complications. Third, by approaching vessel occlusion with a longitudinal approach it is possible to fill the targeted space with either coils or a plug and not actually occlude the vessel. The fourth and final limitation of the technique can occur as a result of trying to avoid point 3: the vessel is not occluded and the operator is running out of space to continue embolizing so they add forward pressure to the coil in order to “pack it” and it, in turn, ruptures the vessel. These are all known complications or limitations to what has always been the standard approach to vascular occlusion.

It is therefore necessary to find an alternative way to occlude vessels. When the body naturally occludes a vessel, it does so from the outside-in via a stenosis. For example, when a patient has a heart attack due to a stenosis, they have a stent inserted to prop open the vessel. Therefore, the body's natural approach to vessel occlusion is not longitudinal, but concentric. The device of the present disclosure, the Cinch Concentric Occlusion Tool, is the first vascular occlusion tool that approaches vessel occlusion in a concentric manner gradually narrowing the vessel from the outside-in until the vessel occludes. By approaching embolization in this manner, all four of the known limitations/complications associated with the longitudinal embolization and the devices associated with it are eliminated.

### SUMMARY

There is a need for implantable and expandable medical devices suitable for occluding fluid flow in a blood vessel. The present disclosure is directed toward further solutions to address this need, in addition to having other desirable characteristics.

According to an embodiment of a present disclosure, there is provided a medical device for occluding a lumen of a body cavity comprising: a proximal end; a distal end; an elongated member positioned between the proximal end and the distal end, wherein the elongated member has a lumen passing through; and a flow-limiting member coupled to the distal end.

According to aspects of the present disclosure, the elongated member comprises an inner surface defining the lumen through the elongated member and an outer surface.

According to aspects of the present disclosure, the elongated member is capable of transitioning between a collapsed configuration and an expanded configuration.

According to aspects of the present disclosure, the elongated member is configured to engage a surface of a body cavity in the expanded configuration.

According to aspects of the present disclosure, the device is formed of a combination of platinum, cobalt chromium, and nitinol.

According to aspects of the present disclosure, the flow-limiting member resides within the lumen.

According to aspects of the present disclosure, the flow-limiting member is movable between a closed and an open configuration.

According to aspects of the present disclosure, the flow-limiting member is configured to substantially prevent a flow of a fluid in the body cavity past the flow-limiting member in the open configuration.

According to aspects of the present disclosure, the flow-limiting member is a cap.

According to aspects of the present disclosure, the cap prevents the flow of a fluid in the body cavity past the flow-limiting member.

According to an embodiment of a present disclosure, there is provided a method for occluding a lumen of a body cavity comprising: providing a medical device comprising: a proximal end; a distal end; an elongated member positioned between the proximal end and the distal end, wherein the elongated member has a lumen passing through; and a flow-limiting member coupled to the distal end; placing the device in the lumen of a body cavity; and expanding the elongated member.

According to aspects of the present disclosure, the elongated member comprises an inner surface defining the lumen through the elongated member and an outer surface.

According to aspects of the present disclosure, the elongated member is capable of transitioning between a collapsed configuration and an expanded configuration.

According to aspects of the present disclosure, the elongated member is configured to engage a surface of a body cavity in the expanded configuration.

According to aspects of the present disclosure, the device is formed of a combination of platinum, cobalt chromium, and nitinol.

According to aspects of the present disclosure, the flow-limiting member resides within the lumen.

According to aspects of the present disclosure, the flow-limiting member is movable between a closed and an open configuration.

According to aspects of the present disclosure, the flow-limiting member is configured to substantially prevent a flow of a fluid in the body cavity past the flow-limiting member in the open configuration.

According to aspects of the present disclosure, the flow-limiting member is a cap.

According to aspects of the present disclosure, the cap prevents the flow of a fluid in the body cavity past the flow-limiting member.

### BRIEF DESCRIPTION OF THE FIGURES

These and other characteristics of the present disclosure will be more fully understood by reference to the following detailed description in conjunction with the attached drawings, in which:

FIG. 1 is a perspective view of an occluding medical device in accordance with an embodiment of the present disclosure;

FIG. 2 is a perspective view of an occluding medical device in accordance with an embodiment of the present disclosure;

FIG. 3 is a perspective view of an occluding medical device in accordance with an embodiment of the present disclosure;

FIG. 4A, FIG. 4B, and FIG. 4C is a perspective view of an occluding medical device in accordance with an embodiment of the present disclosure;

FIG. 5A, FIG. 5B, FIG. 5C, and FIG. 5D is a perspective view of an occluding medical device in accordance with an embodiment of the present disclosure; and

### DETAILED DESCRIPTION

An illustrative embodiment of the present disclosure relates to implantable and expandable medical devices suitable for occluding fluid flow in a blood vessel, and specifically to occluding medical devices that are capable of embolizing vessels from a concentric, outward-in approach rather than a traditional longitudinal approach.

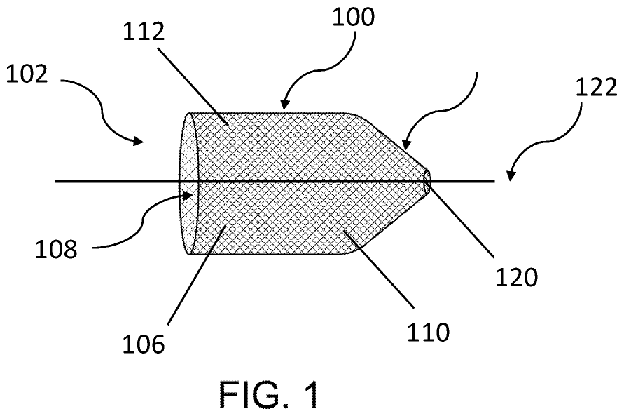

FIGS. 1 through FIG. 5, wherein like parts are designated by like reference numerals throughout, illustrate an example embodiment or embodiments of vessel occluding devices, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property. As referred to herein, the terms “proximal” and “distal” are in relation to the delivery handle of the stent delivery system (also referred to as a catheter). For example, the distal end 104 of the occluding device 100 is the end that is inserted first into a body of a patient and the proximal end 102 is opposite the distal end 104. As used herein, the term “about” refers to a ten percent range around a specified number. For instance, “about 10” refers to a range from 9.9 to 10.1.

FIG. 1 illustrates an embodiment of an occluding device 100 of the present disclosure. As used herein, the terms “occluding” and “occlusion” refer interchangeably to partial or completion blocking of the body cavity into which the device is deployed. The occluding device 100 is shown in an expanded configuration.

In some embodiments, the occluding device 100 includes a proximal end 102, a distal end 104, and an elongated member 106 extending between the proximal end 102 and distal end 104. The elongated member 106 is capable of being expanded from a compressed position (e.g., during delivery) to an expandable position (e.g., once deployed). The elongated member 106 can be self-expanding or balloon-expandable, or sized by any methods known to those of skill in the art. The elongated member 106 forms a scaffold structure that is generally tubular in shape and has a lumen 108 within the scaffold structure.

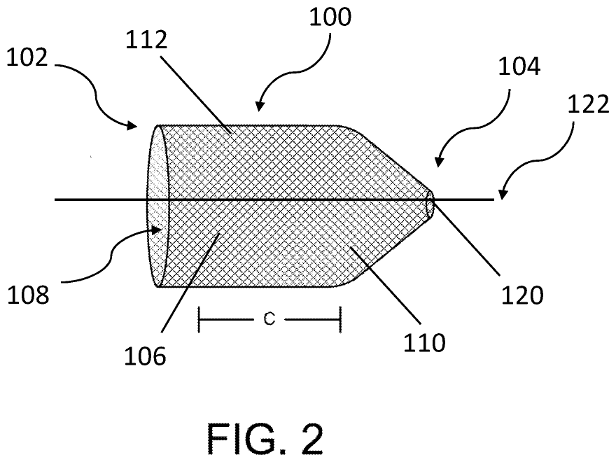

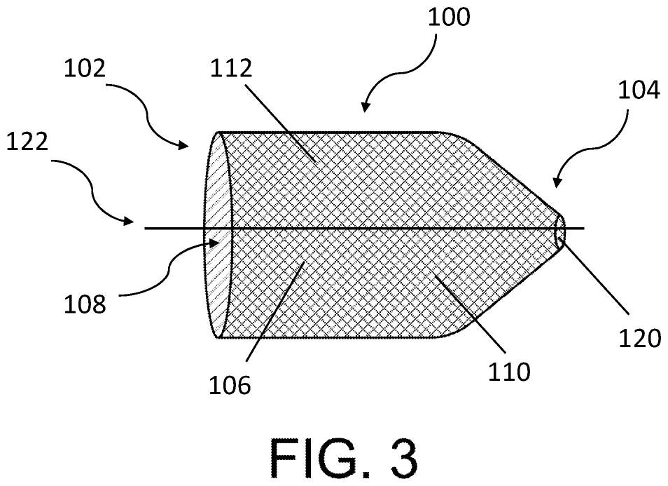

The elongated member 106 further comprises a web structure 110 that is configured to allow the occluding device 100 to expand from a constrained, collapsed, or contracted delivery configuration to an expanded deployed configuration. In an embodiment, the web structure 110 can have a braided design. As shown in FIG. 1, FIG. 2, and FIG. 3, the elongated member 106 is formed by having a plurality of elongate wires 112 formed into the web structure 110. The elongate wires 112 traverse the length of the elongated member 106 in a direction traverse to the longitudinal length of the elongated member 106. The elongate wires 112 may be formed into the web structure 108 by braiding the wires 112, winding the wires 112, knitting the wires 112, and combinations thereof. In one embodiment, the wires 112 are braided in a braided pattern to form the web structure 108. A useful nonlimiting braided pattern includes a one over and one under pattern, but other patterns may suitably be used. knitting wires or wire filaments into a braided loop, the pattern of which loops are formed from a relaxed state, where each row of loops is axially and independently of the rows on either side.

The elongated member 106 of the present disclosure can act similarly to a stent. Stents are commonly used as supporting structure in cardiovascular and other vessel related procedures, for example, as a support as well as to maintain vessel patency after a balloon angioplasty procedure. Advantageously, a stent can be advanced within the lumen of a body cavity, for example, within a vessel, in a compressed or crimped conformation. Once in place the stent is either expanded or allowed to expand such that the device contacts a surface of the body cavity. In some embodiments the surface is an inner surface of the body cavity. In some embodiments, the body cavity is a vessel, for example, a blood vessel, and the device contact an inner surface of the vessel when deployed.

Furthermore, occlusion devices 100 of the present disclosure preferably exhibit high radial stiffness in the deployed configuration. Occlusion devices therefore are capable of withstanding compressive forces applied by a vessel wall, thereby alleviating stenosis and maintaining vessel patency. The web structure 108 of the present disclosure provides the desired combination of flexibility in the delivery configuration to allow for safe navigation and radial stiffness in the deployed configuration to allow for excellent apposition to the vessel wall. In addition, the flexibility of the web structure 108 of the present disclosure allows the occlusion device 100 to conform to the anatomy of the intracranial veins.

The occlusion device 100 of the present disclosure may be made of a combination of suitable materials, including platinum, cobalt chromium, nitinol alloy (also known as nickel titanium), polyester or a combination of materials. Since the occlusion device 100 of the present disclosure is self-expanding, the web structure 108 in an embodiment is fabricated from an elastic material. In accordance with further embodiments of the present disclosure, occlusion device 100 is fabricated from biocompatible and/or biodegradable materials. Biocompatible material may comprise a biocompatible polymer, for example, a modified thermoplastic Polyurethane, Polyethylene Terephthalate, Polyethylene Tetraphthalate, expanded Polytetrafluoroethylene, Polypropylene, Polyester, Nylon, Polyethylene, Polyurethane, or combinations thereof. In an embodiment, the occlusion device 100 of the present disclosure is constructed of about 80% cobalt chromium and about 20% platinum filaments of various sizes.

The occlusion device 100 of the present disclosure may be sized to cover the anatomy of different blood vessels. For example, depending on the area of need, the length of the occlusion device 100 may be between about 10 mm to about 80 mm long. In an embodiment, the length of the occlusion device 100 is between about 1 mm to about 5 mm. In an embodiment, the length of the occlusion device 100 is between about 1 mm to about 10 mm. In an embodiment, the length of the occlusion device 100 is between about 1 mm to about 15 mm. In an embodiment, the length of the occlusion device 100 is between about 1 mm to about 20 mm. In an embodiment, the length of the occlusion device 100 is between about 1 mm to about 25 mm. In an embodiment, the length of the occlusion device 100 is between about 1 mm to about 30 mm. In an embodiment, the length of the occlusion device 100 is between about 1 mm to about 35 mm. In an embodiment, the length of the occlusion device 100 is between about 1 mm to about 40 mm. In an embodiment, the length of the occlusion device 100 is between about 1 mm to about 45 mm. In an embodiment, the length of the occlusion device 100 is between about 1 mm to about 50 mm. In an embodiment, the length of the occlusion device 100 is between about 1 mm to about 55 mm. In an embodiment, the length of the occlusion device 100 is between about 1 mm to about 60 mm. In an embodiment, the length of the occlusion device 100 is between about 1 mm to about 65 mm. In an embodiment, the length of the occlusion device 100 is between about 1 mm to about 70 mm. In an embodiment, the length of the occlusion device 100 is between about 1 mm to about 75 mm. In an embodiment, the length of the occlusion device 100 is between about 1 mm to about 80 mm. In an embodiment, the length of the occlusion device 100 is between about 5 mm to about 10 mm. In an embodiment, the length of the occlusion device 100 is between about 5 mm to about 15 mm. In an embodiment, the length of the occlusion device 100 is between about 5 mm to about 20 mm. In an embodiment, the length of the occlusion device 100 is between about 5 mm to about 25 mm. In an embodiment, the length of the occlusion device 100 is between about 5 mm to about 30 mm. In an embodiment, the length of the occlusion device 100 is between about 5 mm to about 35 mm. In an embodiment, the length of the occlusion device 100 is between about 5 mm to about 40 mm. In an embodiment, the length of the occlusion device 100 is between about 5 mm to about 45 mm. In an embodiment, the length of the occlusion device 100 is between about 5 mm to about 50 mm. In an embodiment, the length of the occlusion device 100 is between about 5 mm to about 55 mm. In an embodiment, the length of the occlusion device 100 is between about 5 mm to about 60 mm. In an embodiment, the length of the occlusion device 100 is between about 5 mm to about 65 mm. In an embodiment, the length of the occlusion device 100 is between about 5 mm to about 70 mm. In an embodiment, the length of the occlusion device 100 is between about 5 mm to about 75 mm. In an embodiment, the length of the occlusion device 100 is between about 5 mm to about 80 mm. In an embodiment, the length of the occlusion device 100 is between about 10 mm to about 15 mm. In an embodiment, the length of the occlusion device 100 is between about 10 mm to about 20 mm. In an embodiment, the length of the occlusion device 100 is between about 10 mm to about 25 mm. In an embodiment, the length of the occlusion device 100 is between about 10 mm to about 30 mm. In an embodiment, the length of the occlusion device 100 is between about 10 mm to about 35 mm. In an embodiment, the length of the occlusion device 100 is between about 10 mm to about 40 mm. In an embodiment, the length of the occlusion device 100 is between about 10 mm to about 45 mm. In an embodiment, the length of the occlusion device 100 is between about 10 mm to about 50 mm. In an embodiment, the length of the occlusion device 100 is between about 10 mm to about 55 mm. In an embodiment, the length of the occlusion device 100 is between about 10 mm to about 60 mm. In an embodiment, the length of the occlusion device 100 is between about 10 mm to about 65 mm. In an embodiment, the length of the occlusion device 100 is between about 10 mm to about 70 mm. In an embodiment, the length of the occlusion device 100 is between about 10 mm to about 75 mm. In an embodiment, the length of the occlusion device 100 is between about 10 mm to about 80 mm. In an embodiment, the length of the occlusion device 100 is between about 40 mm to about 45 mm. In an embodiment, the length of the occlusion device 100 is between about 45 mm to about 50 mm. In an embodiment, the length of the occlusion device 100 is between about 50 mm to about 55 mm. In an embodiment, the length of the occlusion device 100 is between about 55 mm to about 60 mm. In an embodiment, the length of the occlusion device 100 is between about 60 mm to about 65 mm. In an embodiment, the length of the occlusion device 100 is between about 65 mm to about 70 mm. In an embodiment, the length of the occlusion device 100 is between about 70 mm to about 75 mm. In an embodiment, the length of the occlusion device 100 is between about 75 mm to about 80 mm. As used herein, the term “about” or “approximately” refers to a variation of 10% from the indicated values (e.g., 40 mm, 45 mm, 50 mm, etc.), or in case of a range of values, means a 10% variation from both the lower and upper limits of such ranges. For instance, “about 40 mm” refers to a range of between 36 mm and 44 mm.

It should be appreciated that the length of the occlusion device 100 can depend on the artery in which it is positioned. For instance, the length of the occlusion device 100 is between about 1 mm to about 5 mm in a gastric artery. In an embodiment, the length of the occlusion device 100 is between about 3 mm to about 30 mm in a splenic artery. In an embodiment, the length of the occlusion device 100 is between about 5 mm to about 20 mm in an internal iliac artery. In an embodiment, the length of the occlusion device 100 is between about 10 mm to about 40 mm in an internal carotid artery. In an embodiment, the length of the occlusion device 100 is between about 20 mm to about 40 mm in a common hepatic artery. In an embodiment, the length of the occlusion device 100 is between about 1 mm to about 10 mm in a middle meningeal artery. In an embodiment, the length of the occlusion device 100 is between about 3 mm to about 7 mm in a pudendal artery. In an embodiment, the length of the occlusion device 100 is between about 3 mm to about 20 mm in a pulmonary artery. Depending on the area of treatment, the diameter of the occlusion device 100 may be between about 0.5 mm to about 10 mm. In an embodiment, the diameter of the occlusion device 100 is between about 1 mm and about 9.5 mm. In an embodiment, the diameter of the occlusion device 100 is between about 1.5 mm and about 9 mm. In an embodiment, the diameter of the occlusion device 100 is between about 2 mm and about 8.5 mm. In an embodiment, the diameter of the occlusion device 100 is between about 2.5 mm and about 8 mm. In an embodiment, the diameter of the occlusion device 100 is between about 3 mm and about 7.5 mm. In an embodiment, the diameter of the occlusion device 100 is between about 3.5 mm and about 7 mm. In an embodiment, the diameter of the occlusion device 100 is between about 4 mm and about 6.5 mm. In an embodiment, the diameter of the occlusion device 100 is between about 4.5 mm and about 6 mm. In an embodiment, the diameter of the occlusion device 100 is between about 5 mm and about 5.5 mm.

It should be appreciated that the diameter of the occlusion device 100 can depend on the artery in which it is positioned. For instance, the diameter of the occlusion device 100 is between about 0.5 mm and about 2 mm in a spinal artery. In an embodiment, the diameter of the occlusion device 100 is between about 5 mm and about 8 mm in a gastroduodenal artery. In an embodiment, the diameter of the occlusion device 100 is between about 4 mm and about 7 mm in an internal carotid artery. In an embodiment, the diameter of the occlusion device 100 is between about 5 mm and about 10 mm in a splenic artery.

The elongated member 106 is adapted to serve as a framework for a flow-limiting member 120. The flow-limiting member 120 acts substantially like a cinching mechanism to cut off, or occlude, blood flow. In an embodiment, the flow-limiting member 120 is movable between a closed configuration (e.g., upon insertion) and an open configuration (e.g., once deployed). Once deployed, the occluding device 100 can have a partially, substantially, or completely narrowed distal end 104 permitting distal access while have a proximal end 102 that is fully open or apposed (e.g., against) to the vessel wall. The designed allowed for successive devices to be placed inside one another thus embolizing the vessel concentrically instead of longitudinally. Additionally, the embodiments of the present disclosure may serve as a backstop to other embolic materials in both antegrade and retrograde fashion.

In some embodiments, the flow-limiting member 120 is found on the distal end 104 of the elongated member 106. In some embodiments (not illustrated), the flow-limiting member 120 can be found at other locations on the elongated member 106. In some embodiments a plurality of flow-limiting members can be used. For example, in some embodiments, two flow-limiting members can be used. Where two such members are used, a flow-limiting member can be placed both over and within the expandable member, both can be placed within the member, or both can be placed over the expandable member. In some embodiments, a plurality of flow-limiting members can be more than two. All such combinations and configurations are contemplated with be within the scope of the present disclosure. In some embodiments, there can be at least one space between two of the plurality of flow-limiting members, said at least one space useful to accommodate a bioactive agent or other substances useful to promote healing, occlusion, or attachment of the device to a surface of the body cavity engaged by the device.

The elongated member 106 and flow-limiting member 120 act cooperatively, such that when the elongated member 106 is expanded within a body cavity to be occluded, the flow-limiting member 120 can remain entirely or partially cinched. In other words, when the occluding device 100 substantially fills the entire vessel lumen, the flow-limiting member 120 can effectively occlude flow through the vessel.

It should be appreciated that different variations of the flow-limiting member 120 may exist. In one embodiment, the flow-limiting member 120 may have a partial opening on the distal end 104 to allow wire/catheter access beyond the targeted site of occlusion. Such a design can also allow subsequent, sequential, or additional occluding devices 100 to be placed inside the original design. In an alternative embodiment, the flow-limiting member 120 may have a cap at the distal end 104 instead of an opening on. In this variation, the distal end 104 is closed or capped and the hole is eliminated in favor of additional braiding down the lumen of the vessel. In some embodiments, variations that include this cap may also include dacron fiber or other thrombogenic material at the distal end 104 to enhance occlusion effectiveness or thrombogenicity. In yet another embodiment, the flow-limiting member 120 may be found at both the distal end 104 of the occluding device 100 as well as in one of my places along the elongated member 106. This design may be used in situations where long segments of vessels are being occluded.

The different variations of the occlusion devices 100 are shown in FIG. 1 through FIG. 6. As shown in FIG. 1, the flow-limiting member 120 may have a partial opening on the distal end 104 to allow wire/catheter 122 access beyond the targeted site of occlusion. In an embodiment, the flow-limiting member 120 may have an opening of about 2.6 mm to about 0.867 mm. The occluding device 100 of this embodiment may be constructed of 64 strands approximately sized between about 26 microns to about 34 microns each and deliverable through a microcatheter with a lumen of about 0.027 inches in diameter. The catheter wire may be constructed of a 0.016 inch nitinol wire that is 180 cm in length and have a semi-retrievable attachment to the occluding device 100. In an embodiment, the occluding device 100 will open to about 0.5 mm greater than it is sized.

FIG. 2 shows yet another embodiment of the flow-limiting member 120 having a partial opening on the distal end 104 to allow wire/catheter 122 access beyond the targeted site of occlusion. In this embodiment, the flow-limiting member 120 may have an opening of about 1.8 mm to about 0.6 mm. The occluding device 100 of this embodiment may be constructed of 48 strands approximately sized between about 22 microns to about 30 microns each and deliverable through a microcatheter with a lumen of about 0.017 inches in diameter. The catheter wire may be constructed of a 0.013 inch nitinol wire that is 180 cm in length and have a semi-retrievable attachment to the occluding device 100. In an embodiment, the occluding device 100 will open to about 0.5 mm greater than it is sized.

FIG. 3 shows another embodiment of the flow-limiting member 120 having a partial opening on the distal end 104 to allow wire/catheter 122 access beyond the targeted site of occlusion. In this embodiment, the flow-limiting member 120 may have an opening of about 1.5 mm to about 0.5 mm. The occluding device 100 of this embodiment may be constructed of 32 strands approximately sized between about 18 microns to about 26 microns each and deliverable through a microcatheter with a lumen of about 0.013 inches in diameter. The catheter wire may be constructed of a 0.010 inch nitinol wire that is 180 cm in length and have a semi-retrievable attachment to the occluding device 100. In an embodiment, the occluding device 100 will open to about 0.5 mm greater than it is sized.

In an embodiment, the distal end 104 may be narrowed with or without a leading-edge nose cone and it may be constrained with an external mechanism such as a laser cut stent or scaffold or a platinum coil. Such nose cone may be up to 1 mm in length. In another embodiment, the distal end 104 may be set to a predetermined diameter through a simple heat setting process.

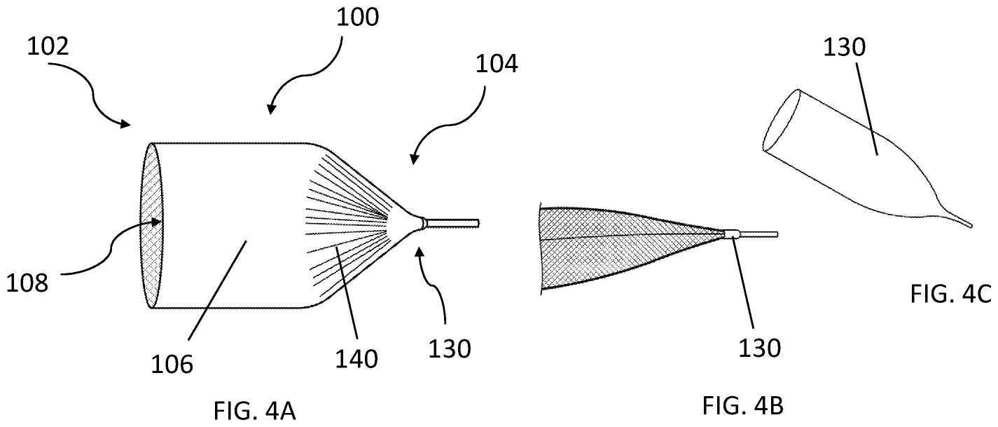

In an embodiment shown in FIG. 4A, FIG. 4B, and FIG. 4C, the flow-limiting member 120 may have a cap or shuttlecock design. In this embodiment, the flow-limiting member 120 is closed and, optionally, bound by a cap 130 that prevents access distally. Inside the cap 130, there may be fibers 140 that aid with enhancing the occluding effectiveness of the occlusion device 100. In an embodiment, the fibers 140 may be thrombogenic dacron fibers. The embodiment shown in FIG. 4A may alternatively consist of a single or double layer of braiding tethered to each other via distal or leading-edge crimp or series of crimps, which effectively closes the distal end 104. The crimps may be inserted at specific intervals within the elongated member 106 of the occluding device 100 or it may include a series of rigid shuttlecocks. These shuttlecocks provide the structure for what is the leading edge of a series of football or sausage shapes. This inner shuttlecock props open the leading edge of the crimped outer stent. The trailing portion (about 50%) of the outer stent then can be folded inside the first portion (about 50%) of the preceding stent. The layers of braiding may consist or any combination of the nitinol, cobalt chromium, and polyester, but other suitable materials may also be used. The remaining features of this embodiment may be similar to those described above. In an embodiment, where there are two or more layers of braiding, there may be material or fabric between them which is adhered to the layers via a crimp. In an embodiment, the material or fabric may be PTFE, nylon, polyester, or any other suitable material or fabric.

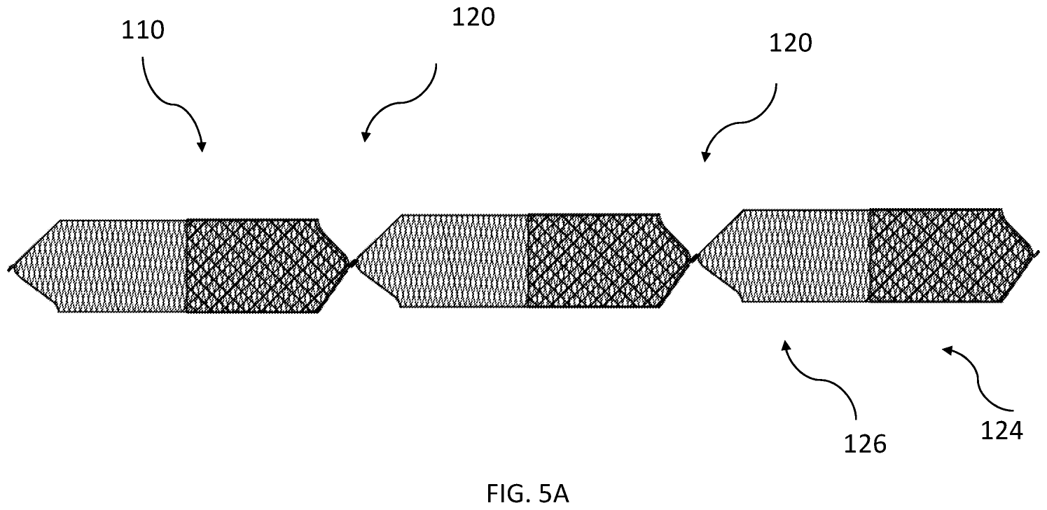

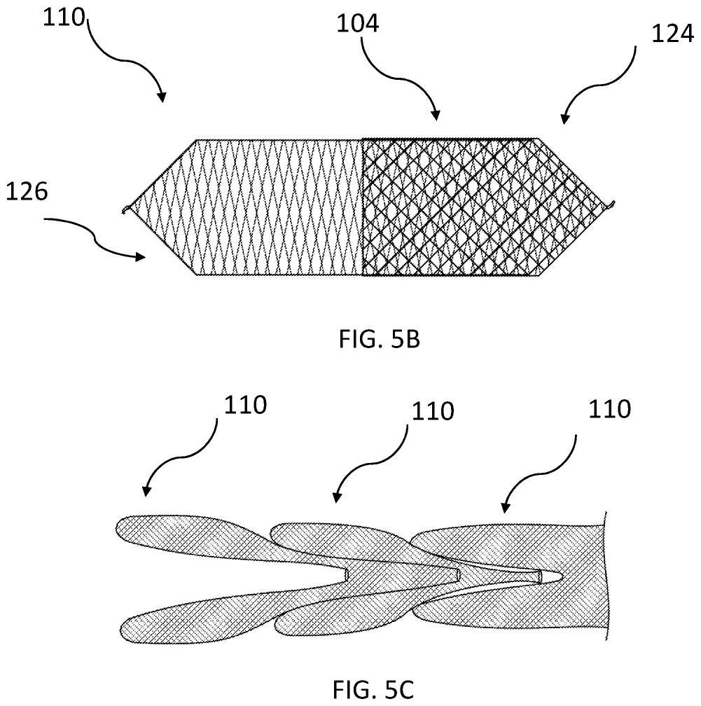

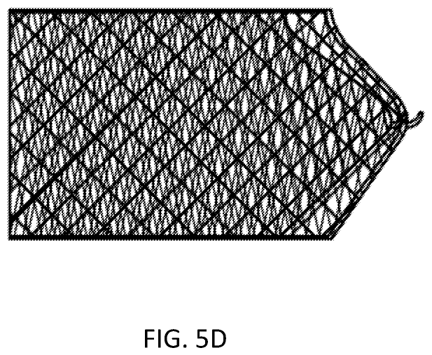

In an embodiment shown in FIG. 5A, FIG. 5B, FIG. 5C, and FIG. 5D two or more occluding devices 100 may be connected, stacked, packed, or layered together in a packed or sequential manner. In an embodiment, occluding devices 110 having the shuttlecock design of FIG. 4A, FIG. 4B, and FIG. 4C may be used to accomplish this stacked design. This allows for concentric embolization to occur without having to use single or individual occluding devices 110.

FIG. 5A shows this embodiment in its extended state with the flow-limiting members 120 connecting two occluding devices 110 to create a single cylindrical braided stent. In an embodiment, the occluding device 110 is made from a single structure and is cinched or crimped in places to form the flow limiting member 120. Crimping the occluding device 110 at multiple points allows the connecting of two layers of braids and also effectively creates several sausage links or football shapes. In another embodiment, the occluding device 110 is made from connecting (either reversibly or permanently) the flow limiting members 120 or sequential occluding devices 110.

To form the packed design, a delivery microcatheter works to intussuscept or invaginate the flow-limiting members 120 of one occluding device 110 into the adjacent occluding device 110 from the distal end 104 as shown in FIG. 5B. To accomplish this, the proximal half of the adjacent occluding device 110 (the distal device) is able to be folded over and inside the distal half of the newly inserted occluding device 110. Each successive occluding device 110 has a slightly smaller outer diameter such that it will fit inside of the previously delivered (i.e., more distal) occluding device 110. In other words, this embodiment is a series of connected braided devices that fold inside of one another. The distal side of each occluding device 110 features the dual or double layer of braid 124 while the proximal side is only supported by a single layer of braid 126. This device may consist of a single, double, or multiple layer of braiding tethered to each other via distal and proximal edge crimps which effectively marries the two layers of braid to each other. This outer braid will impart minimal structure or outward radial force. This outer braid will serve as an embolization sock of sorts once the inner series of stents are added. Inside the outer braid, a series of rigid braided stents will be inserted and then affixed to the outer stent via a crimp or other mechanism on the proximal and distal edges. The inner braid is intended to be about 30% to about 50% the length of the total length or outer braid. When designed in this manner, the proximal portion (about 50%) of the outer braid can be folded over and then inside (intussuscepted) the robust, structured inner stent because it only extends about 50% of the total length of each football/sausage shape. In an embodiment, the outer braid connects each of the inner braids. The resulting device creates a single embolization device that is made from one or more occluding devices 110 and which is segmented by one or more flow-limiting members 120 and which has sections of single layer braid coverage 126 and others with double layered braid 124 coverage as shown as FIG. 5B and FIG. 5C. FIG. 5D shows this embodiment in its collapsed state.

The embodiment illustrated in FIG. 5A, FIG. 5B, FIG. 5C, and FIG. 5D allows for a user to customize treatment depending on the vessel that needs occluding. If the vessel is large, this embodiment allows you to use multiple occluding devices 110 to occlude the vessel. Conversely, if the vessel is small only one or two occluding devices 110 may be necessary. FIG. 5C shows three concentrically packable occluding devices. This allows a user to sequentially occlude an artery or vein. Alternatively, an embodiment of the present invention may serve as an anchor and/or backstop for other embolization devices such as coils, plugs, or viscous liquid embolics. Likewise, an embodiment of the present invention may partially obstruct flow in a vessel in order to induce collateralization and/or increase pressures distal to the narrowing for the purposes of liquid embolization.

The occlusion device 100 of the present disclosure described above generally have a high degree of compliance, which may or may not include elasticity. These flow-limiting members can be formed from a plurality of filaments that are either tightly woven to occlude fluid flow, or are thrombogenic to promote clotting of the fluid flow, and thereby occlude the flow. In one embodiment, the flow-limiting members 120 may be constructed from the same materials as the elongated member 106. In another embodiment, the flow-limiting members 120 may be constructed from other, more highly thrombogenic, materials. Suitable materials for the plurality of filaments include polyethylene and polyethylene terephthalate. Alternatively, these flow-limiting members can be formed from a sheet of flexible material that is non-porous, is thrombogenic, or is covered with a non-porous coating to occlude fluid flow. Suitable materials for a sheet of flexible material used to form the flow-limiting members include silicone, polyurethane, polycarbonate urethane, polytetrafluoroethylene, or expanded polytetrafluoroethylene.

In some embodiment, occlusion device 100 may contain an external coating or attached active groups C configured for localized delivery of radiation, gene therapy, medicaments, thrombin inhibitors, or other therapeutic agents. For example, occlusion device 100 may be coated with therapeutic agents to help prevent or delay thrombus formation or restenosis within a vessel. Coatings or active groups C may, in an embodiment, be absorbed or adsorbed onto the surface, may be attached physically, chemically, biologically, electrostatically, covalently, or hydrophobically, or may be bonded to the surface through VanderWaal's forces, or combinations thereof, using a variety of techniques that are well-known in the art.

In operation, occlusion device 100 can be delivered over a guide wire via a small incision and the use of fluoroscopic guidance. To deliver the occlusion device 100 to its desired location, the occlusion device 100, in a collapsed configuration, is first inserted into a delivery catheter, then guided through the veins with the help of the guide wire. Once at the desired location, the delivery catheter can be pulled back to expose the occlusion device 100. Upon exposure, the occlusion device 100 self-expands and secures itself to the surrounding vessel walls. In some cases, the system will include an expandable member, such as an inflatable balloon which is used to expand the occlusion device 100 and flow-limiting member 120 to fit up against a vessel wall.

With any embodiment, the occlusion device 100 may be inserted into any peripheral and/or neurovascular vessels which include, without limitation, the gastroduodenal artery, gastric artery, splenic artery, hepatic artery (right or left), pulmonary artery, prostate artery, pudenal artery, uterine artery, renal arteries, internal iliac artery, internal carotid artery, middle meningeal artery, spinal arteries, and anterior cerebral artery.

With any embodiment, the occlusion device 100 may be used for a number of purposes including to maintain patency of blood vessel. Conditions that are suited to be treated using the occlusion device 100 of the present disclosure include, without limitation, gastrointestinal (GI) bleeds, trauma-related hemorrhages anywhere in the body (spleen, kidney, etc.), pre-procedure vessel sacrifice of the gastroduodenal artery to prevent non targeted embolization during radio embolization of liver tumors, embolization of the internal illiac prior to placement of stent graft for the treatment of AAA, presurgical embolization to devascularize surgical sites, such as spine or neck tumors, and pulmonary AVM embolization.

The occlusion device 100 of the present disclosure provides several notable advantages over other occlusion devices currently used. First, since the flow-limiting member 120 is part of the occlusion device 100, the occlusion device 100 avoids the problem of having coils (or plugs) migrate further out (distally) such that the vessel is occluded at a level that was not desired, which can cause patient complications. Similarly, the occlusion device 100 avoids having the coils migrate proximally and occlude larger, non-targeted vessels that can also lead to patient complications. Third, by approaching vessel occlusion with a concentric approach rather than a longitudinal approach, the occlusion device 100 avoids the problem of filling the targeted space with either coils or plugs and not actually occluding the vessel. Finally, the occlusion device 100 avoids the problem of the vessel not being occluded and the operator running out of space to continue result in a ruptured vessel.

Other notable advantages of the occlusion device 100 of the present disclosure include its flexibility to obstruct flow and/or to manage/restrict flow to an area, but not necessarily occlude. Likewise, the occlusion device 100 can artificially narrow a vessel in order to create a “wedged” catheter position, thus creating what is described as a pressure cooker technique. The occlusion device 100 of the present disclosure can also be used to obstruct veins as opposed to arteries. Use in this manner would be helpful in backstopping or preventing liquid embolics that are injected from the arterial anatomy from traveling in the venous side of flow. Similarly, the occlusion device 100 can serve as an anti-reflux device to prevent liquid embolics, such as glue, onyx, or particles, from traveling retrograde in arterial injections. These features of the occlusion device 100 are in part due to the unique design of the occlusion device 100 and especially with the varying designs of the flow-limiting member 120.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the disclosure. It is intended that the present disclosure be limited only to the extent required by the appended claims and the applicable rules of law.

It is also to be understood that the following claims are to cover all generic and specific features of the disclosure described herein, and all statements of the scope of the disclosure which, as a matter of language, might be said to fall therebetween.

What is claimed is:

1. A medical device for occluding a lumen of a body cavity comprising:
a proximal end;
a distal end;
an elongated member positioned between the proximal end and the distal end, wherein the elongated member has a lumen passing through; and
a flow-limiting member coupled to the distal end.

2. The device of claim 1, wherein the elongated member comprises an inner surface defining the lumen through the elongated member and an outer surface.

3. The device of claim 1, wherein the elongated member is capable of transitioning between a collapsed configuration and an expanded configuration.

4. The device of claim 3, wherein the elongated member is configured to engage a surface of a body cavity in the expanded configuration.

5. The device of claim 1, wherein the device is formed of a combination of platinum, cobalt chromium, and/or nitinol.

6. The device of claim 1, wherein the flow-limiting member is movable between a closed and an open configuration.

7. The device of claim 6, wherein the flow-limiting member is configured to substantially prevent a flow of a fluid in the body cavity past the flow-limiting member in the open configuration.

8. The device of claim 1, wherein the flow-limiting member comprises a cap that prevents the flow of a fluid in the body cavity past the flow-limiting member.

9. The device of claim 1, further comprising a flow-limiting member coupled to the proximal end.

10. The device of claim 9, further comprising two or more medical devices coupled together by flow-limiting member, wherein the proximal end of the first medical device is folded in on itself forming a double layer, and wherein the distal end of the second medical device is inserted into the proximal end of the first medical device.

11. A method for occluding a lumen of a body cavity comprising:
providing a medical device comprising:
a proximal end;
a distal end;
an elongated member positioned between the proximal end and the distal end,
wherein the elongated member has a lumen passing through; and
a flow-limiting member coupled to the distal end;
placing the device in the lumen of a body cavity; and
expanding the elongated member.

12. The method of claim 11, wherein the elongated member comprises an inner surface defining the lumen through the elongated member and an outer surface.

13. The method of claim 11, wherein the elongated member is capable of transitioning between a collapsed configuration and an expanded configuration.

14. The method of claim 13, wherein the elongated member is configured to engage a surface of a body cavity in the expanded configuration.

15. The method of claim 11, wherein the device is formed of a combination of platinum, cobalt chromium, and/or nitinol.

16. The method of claim 11, wherein the flow-limiting member is movable between a closed and an open configuration.

17. The method of claim 16, wherein the flow-limiting member is configured to substantially prevent a flow of a fluid in the body cavity past the flow-limiting member in the open configuration.

18. The method of claim 11, wherein the flow-limiting member comprises a cap that prevents the flow of a fluid in the body cavity past the flow-limiting member.

19. The method of claim 11, further comprising a flow-limiting member coupled to the proximal end.

20. The method of claim 19, further comprising inserting a second medical device into the first medical device, wherein the proximal end of the first medical device is folded in on itself forming a double layer, and wherein the distal end of the second medical device is inserted into the proximal end of the first medical device.
